SEQUENCE LISTING (9 sequences)

SEQ ID NO: 1 - DNA, 1001 bp, Homo sapiens
  variation (1)..(1001): Age-related maculopathy susceptibility 2 (ARMS2), rs10490924 |allelePos=501|alleles=′G/T′.  T is the risk allele.
tacaatcaaggttttttttttttttcaaatccctgggtctctgcattttttaaaagcttcacagatgatttcaatggatactagggacctctgttgcctcctctggcagagcaggactgaggggtggaccctccctgagaccacccaacaattcagggtggagttatcagggcgccctgactcctgggggcatttttgtgtgacgggaaaagacaatgctcctggctgagtgagatggcagctggcttggcaaggggacagcacctttgtcaccacattatgtccctgtaccctacatgctgcgcctatacccaggaccgatggtaactgaggcggaggggaaaggagggcctgagatggcaagtctgtcctcctcggtggttcctgtgtccttcatttccactctgcgagagtctgtgctggaccctggagttggtggagaaggagccagtgacaagcagaggagcaaactgtctttatcacactccatgatcccagcttctaaaatccacactgagctctgcttaccagccttcttctctcctgctggaacccagaggaggttccagcagcctcagcaccacctgacactggtaagaaatgcagatgatcaggccttaccccagacctattgaatcagaaattctggagtggtgccctgcagcttgcattttaaccagccttcaggtgcttctgatgcatgctcaggtttgagcaccactggccacagggaggcctaggcaattcagccttcctctggttgaatagctggagaattgggaatatcagtaaatacttccaatgcacctgctacatgccagaaaaaggaaacaagaagacgcagtaggtctgagaaagtgatggggtgagcagaaacccaaagcttatagaaggccatctgagtggcccctcaagccggtgaattggctttagggtttactgaaggaggtggaaacctcagcctgcttctcgtccgggttgttagaggagtcatttagaaa

SEQ ID NO: 2 - DNA, 501 bp, Homo sapiens
  variation (1)..(501): HtrA serine peptidase 1 (HTRA1) rs11200638|allelePos=251|alleles=′A/G′ G is the risk allele.
tggtgaaaaatcaaaagaggggaaaacccctttcccatctgagaccgctccaccctcgccagttacgagctgccgagccgcttcctaggctctctgcgaatacggacacgcatgccacccacaacaactttttaaaagaatcagacgtgtgaaggattctattcgaattacttctgctctctgcttttatcacttcactgtgggtctgggcgcgggctttctgccagctccgcggacgctgccttcgtccggccgcagaggccccgcggtcagggtcccgcgtgcggggtaccgggggcagaaccagcgcgtgaccggggtccgcggtgccgcaacgccccgggtctgcgcagaggcccctgcagtccctgcccggcccagtccgagcttcccgggcgggcccccagtccggcgatttgcaggaactttccccggcgctcccacgcgaagccgccgcagggcccccttgcaaagttccattagtttgaaggacgcgaatct

SEQ ID NO: 3 - DNA, 1001 bp, Homo sapiens
  variation (1)..(1001): Complement factor H (CFH) rs1061170|allelePos=501|alleles=′C/T′. C is the risk allele.
atgcctcagtaagtaaacctctgaactgctatatatatgtataaaactttcaaagatcgaagaaaggagagcacataagtgattacacctgtcttatgtaacagaaatagggccaagaaaagagttgttcaagcaaagtgaccaaaatagatcttttctattatgagggtttcttcttgaaaatcacaggagaaataaatatagggactttatgagaatatctatataatttatacatctattaattataaaaactaaagataagtaatattgaatattgatatttctttttgtgcaaacctttgttagtaactttagttcgtcttcagttatacattatttttggatgtttatgcaatcttatttaaatattgtaaaaataattgtaatatactattttgagcaaatttatgtttctcatttactttatttatttatcattgttatggtccttaggaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcagtaagtacactactctgaaatcctagcatgttcatgtctttctaagtaacatagatgacattctaagactcatctatattaattgtggcaaaatgtttatgtcaccttgttttaccaatggacctatttagtttttggtttttcaactgtgtataaatgaatatacaaatttcttgataagtacatagtaaaataaattctcctattaatgggcattagtcaagaatacagtaaaagaatttgaacacaatacttgttggttaaatgaagtcgtattgaagcggcatcattgtctgggtaaatatctgaggttcgttatctcacaccaagaagattaaggacgtggacacacacaaggagtgagttta

SEQ ID NO: 4 - protein, 5 aa, Homo sapiens
  DOMAIN (1)..(5): Antibody 8109, HCDR1
SYAIS

SEQ ID NO: 5 - protein, 17 aa, Homo sapiens
  DOMAIN (1)..(17): Antibody 8109, HCDR2 domain
GIGPFFGTANYAQKFQG

SEQ ID NO: 6 - protein, 7 aa, Homo sapiens
  DOMAIN (1)..(7): Antibody 8109, HCDR3
DTPYFDY

SEQ ID NO: 7 - protein, 11 aa, Homo sapiens
  DOMAIN (1)..(11): Antibody 8109, LCDR1 domain
SGDSIPNYYVY

SEQ ID NO: 8 - protein, 7 aa, Homo sapiens
  DOMAIN (1)..(7): Antibody 8109, LCDR2
DDSNRPS

SEQ ID NO: 9 - protein, 11 aa, Homo sapiens
  DOMAIN (1)..(11): Antibody 8109, LCDR3
QSFDSSLNAEV

(19) United States
(12) Patent Application Publication
(10) Pub. No.: US 2017/0073754 A1
(43) Pub. Date: Mar. 16, 2017

(54) IMPACT OF GENETIC FACTORS ON DISEASE PROGRESSION AND RESPONSE TO ANTI-C5 ANTIBODY IN GEOGRAPHIC ATROPHY

(71) Applicants: Yunsheng HE, Waltham, MA (US); Yue Li, Reinach (CH); Parisa ZAMIRI, Cambridge, MA (US)

(72) Inventors: Yunsheng HE, Waltham, MA (US); Yue Li, Reinach (CH); Parisa ZAMIRI, Cambridge, MA (US)

(73) Assignee: Novartis AG, Basel (CH)

(21) Appl. No.: 15/113,003

(22) PCT Filed: Feb. 5, 2015
(86) PCT No.: PCT/US2015/014585

§ 371 Date: Jul. 20, 2016

Related U.S. Application Data
(60) Provisional application No. 61/936,980, filed on Feb. 7, 2014.
(60) Provisional application No. 61/942,263, filed on Feb. 20, 2014.

(51) Int. Cl. C12Q 1/68; C07K 16/18

(52) U.S. Cl. CPC C12Q 1/6883; C07K 16/18; C12Q 2600/156; C12Q 2600/118; C12Q 2600/106; C07K 2317/565; C07K 2317/76

(57) ABSTRACT

Pharmacogenetic analysis revealed an effect of risk alleles in ARMS2 and CFH genes on the response of subjects to anti-C5 antibodies in the treatment of the progression of geographic atrophy.

### FIELD OF THE INVENTION

The invention relates generally to a measuring or testing process involving nucleic acids encoding proteins, and specifically to the use of biomarkers for the progression of geographic atrophy in subjects as a guide to administration of antibodies targeting complement protein C5 to the subjects.

### BACKGROUND OF THE INVENTION

Age-related macular degeneration (AMD) is the leading cause of legal blindness in persons over the age of 65 years (Klein et al. (2006) Ophthalmol. 113(3):373-80). The advanced form of the disease is divided between a “wet” (neovascular) form and a “dry” (geographic atrophy) form. Geographic atrophy (GA) is an advanced atrophic form of dry AMD. GA is characterized by loss of photoreceptors, retinal pigment epithelium (RPE), and choriocapillaris within the macula. GA is therefore considered to be the end stage of AMD in the absence of choroidal neovascularization (CNV) (Sunness et al. (1999) Ophthalmol. 106(9):1768-79). GA lesions grow in a linear fashion, leading to average growth rate of ˜2 mm²/year over four years (Lindblad et al. (2009) Arch. Ophthalmol. 127(9):1168-74). In contrast, the wet form of AMD is characterized by neovascularization, with the new formed blood vessels creating the obstruction to vision.

In people over 85 years of age, the incidence of GA is approximately four times that of wet AMD (Klein (2007) Ophthalmic Epidemiol. 14(4):184-7). The 15-year cumulative incidence of GA in individuals 43 to 86 years of age with early signs is 14%.

In the wet form of AMD, vascular endothelial growth factor (VEGF) is overexpressed which induces the neovascularization. Therefore, increased VEGF expression can be used as a biomarker. Antibodies and antibody fragments directed against VEGF are effective in treatments of wet AMD (Rosenfeld et al. (2006) Ophthalmol. 113(4):623).

In contrast to the wet form of AMD, there is little information on biomarkers for GA. Besides vitamins and nutritional practices of modest value, no therapy currently exists to prevent the progression of GA. Patients with lesions peripheral to the fovea (an area of about 1.8 mm responsible for the most detailed, straight-ahead vision of the macula) can have normal visual acuity, but as the lesion grows and the fovea is involved, the visual acuity is severely affected. By effectively treating GA, the overall rate of blindness in the population can be reduced.

Beginning in 2005, human genetic studies strongly implicated dysregulation of the alternative complement pathway in the pathogenesis of AMD (Hageman et al. (2005) Proc. Natl. Acad. Sci. U.S.A. 102(20): 7227-32; Rivera et al. (2005) Hum. Mol. Genet. 14(21):3227-36; Magnusson et al. (2006) PLoS Med. 3(1):e5; Maller et al. (2006) Nat. Genet. 38(9):1055-9). Several independent laboratories have reported that polymorphisms located in several chromosomal locus encoding genes for the alternative pathway of complement system are associated with an increased risk for developing advanced AMD. These include complement factor H (CFH), complement factor B (CFB), and complement C3, each of which has been confirmed in a large meta-analysis (Fritsche et al. (2013) Nat. Genet. 45:433-2).

A locus at chromosome 10q26 has also been independently associated with a risk for AMD. Because this region comprises at least two genes that are in high linkage disequilibrium (ARMS2 and HTRA1), there has been uncertainty about which gene or genes contribute to AMD. For example, the Fisher laboratory used genome wide meta-scan analysis which combined the linkage results from several studies and pointed to a susceptibility locus on chromosome 10q26. Fritsche reported that in separating wet AMD (neovascularization) and GA, that ARMS2 risk alleles associated with a risk of the wet AMD (OR_{NV}=2.97, OR_{GA}=2.50, p_{difference}=0.0008), while CFH associated with the risk of GA (OR_{NV}=2.34, OR_{GA}=2.80, p_{difference}=0.0006) (Fritsche et al. (2013) Nat. Genet. 45:433-2; Fisher et al. (2005) Hum. Mol. Genet. 14:2257-64).

Seddon demonstrated a significant association between CFH and ARMS2 risk alleles and progression to GA from early or intermediate AMD (Seddon et al. (2009) Invest. Ophtha. 50(5):2044-2053). The association of CFH, ARMS2 and C3 with development of GA was confirmed by Scholl et al. (2009) PLoS One 4(10):e7418). Klein detected an association between an ARMS2 variant and GA growth rate, but not associations for CFH, C2, C3, APOE and TLR3 (Klein et al. (2010) Ophthalmol. 117(8): 1554-1559).

GA remains a large unmet medical need because of its high incidence and the absence of effective treatments. Thus, there is a need in the art both for identifying effective therapies for GA and for identifying patients who can benefit from those therapies.

### SUMMARY OF THE INVENTION

Provided herein is a methodology for using the presence of risk alleles in ARMS2 and CFH genes as specific biomarkers in determining the sensitivity of a patient to anti-C5 antibodies. These risk alleles result in a method of screening for a patient's responsiveness to anti-C5 antibodies. The method of diagnosing a GA patient includes: obtaining a patient sample from a subject suspected of being at risk for GA; screening the sample for the presence of the risk alleles indicative of the progression of GA; comparing the results of the screening to the results from a screening of a control sample; and determining that the presence of the risk alleles in the patient sample increases the subject's risk of progression of GA. The presence of the risk alleles that are screened can comprise at least two ARMS2 risk alleles. Alternatively, the presence of the risk alleles can include risk alleles of ARMS2 and risk alleles of CFH. Here, the presence of at least three risk alleles for ARMS2 and CFH indicates a subject with an increased risk of progression to geographic atrophy.

The predictive value of presence of the at least two ARMS2 risk alleles can also be used in a method of treating a subject. Also provided is a method of treating a subject with geographic atrophy. The steps of the treatment method include: obtaining a sample from a subject suspected of being at risk for the progression of geographic atrophy (GA); screening the sample for the presence of the alleles that are indicative of the progression of GA in a subject; comparing the results of the screening to the results of screening of a control sample; diagnosing whether the progression of the subject's GA will be reduced by the administration of an anti-C5 antibody; and administering the anti-C5 antibody to the subject. The method selects for treatment those subjects who carry two or more risk alleles at the ARMS2 and CFH loci. Those selected can have two ARMS2 risk alleles and no CFH risk alleles. Alternatively, those selected can have two ARMS2 risk alleles and one CFH risk allele, or one ARMS2 risk allele and two CFH risk alleles or two ARMS2 and two CFH risk alleles. The presence of two risk alleles for ARMS2 or at least three risk alleles for ARMS2 and CFH indicates that the patient can benefit from treatment with an anti-C5 antibody.

Lastly, an anti-C5 antibody composition is provided for use in treating the progression of geographic atrophy in a patient selected from a patient population that will benefit from anti-C5 antibody treatment. The patient is selected on the basis of the presence of at least two risk alleles detected in a sample taken from the patient.

### BRIEF DESCRIPTION OF THE DRAWINGS

FIG. 1 is a photograph showing the progression of both slow and fast growing GA via fundus autofluoresence (FAF) analysis.

FIG. 2 is line graph showing the effect of anti-C5 antibody treatment on the progression of GA in a non-segregated patient population after 6 months of treatment, as compared with the effect of sham treatment. Anti-C5 antibody treatment resulted in a 5% reduction in growth rate at 6 months

FIG. 3 is a line graph showing the effect of anti-C5 antibody treatment on the progression of GA in segregated patient populations, as compared with the effect of sham treatment. The patients with two risk alleles in ARMS2 demonstrated a 42% reduction in the rate of GA progression at month 6 and 69% at 12 months after treatment with an anti-C5 antibody.

FIG. 4 is a line graph showing the effect of anti-C5 antibody treatment on the progression of geographic atrophy in a segregated patient population, as compared with the effect of sham treatment. The patients with at three risk alleles in the ARMS2 and CFH genes responded to anti-C5 antibody treatment with a 27% reduction in the rate of geographic atrophy lesion growth at 6 months and 29% at 12 months.

FIG. 5A and FIG. 5B are waterfall plots showing the differences in anti-C5 antibody treatment on patients with 2 risk alleles in ARMS2 (5A)(upper graph) and at least 3 risk alleles in ARMS2 and CFH (5B) (lower graph).

FIG. 6 depicts a graph wherein anti-C5 antibody treated patients show a gain in visual acuity of 2 letters when compared to sham operated controls.

### DETAILED DESCRIPTION OF THE INVENTION

The impact of GA lesion growth on visual acuity and visual function varies depending on what area of the retina is affected (Sunness et al. (2008) J. Vis. Impair. Blind. 102(10):600-610). Patients with lesions peripheral to fovea (an area of about 1.8 mm responsible for the most detailed, straight-ahead vision of the macula) can have normal visual acuity. However, as the lesion grows and fovea is involved, the visual acuity is severely affected. Thus, the methods of diagnosis and methods of treatment described here can reduce the progression of GA, preserve visual acuity and function and prevent blindness.

The following methods are:
A method of diagnosing the progression of geographic atrophy (GA) in a subject, the method comprising:
(a) obtaining a sample from a subject;
(b) screening the sample for risk alleles selected from alleles in the group of genes consisting of ARMS2 and CFH;
(c) comparing the results of the screening to the results from a screening of an allele control sample; and
(d) determining that the presence of the risk alleles in the sample indicates an increase in the subject's risk of progression of GA, wherein either:
   (i) the presence of at least two ARMS2 risk alleles is determined to be indicative of the progression of GA in the subject; or
   (ii) the presence of at least three risk alleles selected from the group of genes consisting of ARMS2 and CFH is determined to be indicative of the progression of GA in the subject.
The method wherein the presence of at least two ARMS2 risk alleles is determined to be indicative of the progression of GA in the subject.
The method wherein the ARMS2 risk allele comprises rs10490924 (SEQ ID NO. 1).
The method wherein the presence of at least three risk alleles selected from the group of genes consisting of ARMS2 and CFH is determined to be indicative of the progression of GA in the subject.
The method wherein the ARMS2 risk allele comprises rs10490924 (SEQ ID NO. 1) for ARMS2 and rs1061170 (SEQ ID NO.3) for CFH.
The method wherein the subject's sample is selected from the group consisting of blood sample, blood-derived product, buccal swab, hair root and leukocyte sample.
The method wherein the subject has GA.
The method wherein the screening step (b) is a single nucleotide polymorphism (SNP) assay.
The method wherein the control sample is from one or more subjects without GA.
A method of treating a subject with geographic atrophy (GA), the method comprising;
(a) obtaining a sample from a subject with GA;
(b) screening the sample for the presence of risk alleles selected from alleles in the group of genes consisting of ARMS2 and CFH, wherein the presence of the risk alleles is indicative of the subject's responsiveness to an anti-C5 antibody; (c) comparing the results of the screening to a control sample;
(d) determining whether the subject's GA will be reduced by the administration of an anti-C5 antibody, wherein:
   (i) the presence of at least two ARMS2 risk alleles is indicative of the subject's responsiveness to an anti-C5 antibody; or
   (ii) the presence of at least three risk alleles selected from the group of genes consisting of ARMS2 and CFH is indicative of the subject's responsiveness to an anti-C5 antibody; and
(e) administering the anti-C5 antibody to the subject.
The method wherein the anti-C5 antibody is antibody 8109 (SEQ ID NOs 4-9).
The method wherein the anti-C5 antibody is administered intravitreally to the subject.
The method wherein the presence of at least two ARMS2 risk alleles is indicative of subject's responsiveness to anti-C5 antibodies.
The method wherein the ARMS2 risk allele comprises rs10490924 (SEQ ID NO. 1).
The method wherein the presence of at least three risk alleles of ARMS2 and CFH is indicative of the subject's responsiveness to anti-C5 antibodies.
The method wherein the ARMS2 risk allele comprises rs10490924 (SEQ ID NO. 1) for the ARMS2 gene and rs1061170 (SEQ ID NO. 3) for the CFH gene.
A composition comprising an anti-C5 antibody for use in treatment of progression of geographic atrophy (GA) in a subject with GA, wherein the subject is selected for anti-C5 antibody treatment by the presence of two ARMS2 risk alleles (SEQ ID NO. 1).
A composition comprising an anti-C5 antibody for use in treatment of progression of geographic atrophy in a subject with GA, wherein the subject is selected for anti-C5 antibody treatment by the presence of at least three risk alleles in ARMS2 (SEQ ID NO. 1) and CFH (SEQ ID NO. 3).
The composition wherein the anti-C5 antibody is antibody 8109 (SEQ ID NOs. 4-9).

### DEFINITIONS

“About” in relation to a numerical value x means, for example, x±10%.

“Allele” or “alleles” means one of two versions of a gene or genetic locus. If the two alleles are the same, the individual is homozygous for that gene. If the alleles are different, the individual is heterozygous. Allele includes genes that encode a protein as well as non-coding DNA sequences. “Risk allele” means an allele containing a variation or polymorphism associated with GA. For example, rs10490924 is a risk allele in the ARMS2 gene associated with GA.

An “antibody” includes whole antibodies and any antigen binding fragment (i.e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

An “antigen binding portion” of an antibody refers to one or more fragments of an intact antibody that retain the ability to specifically bind to a given antigen (e.g., C5). Antigen binding functions of an antibody can be performed by fragments of an intact antibody. Examples of binding fragments include a Fab fragment, a monovalent fragment consisting of the VL, VH, CL and CH1 domains; a F(ab)₂ fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al. (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv) (See, e.g., Bird et al. (1988) Science 242:423-426; and Huston et al. (1988) Proc. Natl. Acad. Sci. U.S.A. 85:5879-5883). Such single chain antibodies include one or more “antigen binding portions” of an antibody. These antibody fragments are obtained using conventional techniques known to those of skill in the art, and the fragments are screened for utility in the same manner as are intact antibodies. Antigen binding portions can also be incorporated into single domain antibodies, maxibodies, minibodies, intrabodies, diabodies, triabodies, tetrabodies, v-NAR and bis-scFv (Hollinger and Hudson (2005) Nat. Biotechnol. 23 (9): 1126-1136). Antigen binding portions of antibodies can be grafted into scaffolds based on polypeptides such as fibronectin type III (Fn3). See U.S. Pat. No. 6,703,199, which describes fibronectin polypeptide monobodies. Antigen binding portions can be incorporated into single chain molecules comprising a pair of tandem Fv segments (VH-CH1-VH-CH1) which, together with complementary light chain polypeptides, form a pair of antigen binding regions (Zapata et al. (1995) Protein Eng. 8(10):1057-1062; and U.S. Pat. No. 5,641,870).

“Affinity” means the strength of interaction between antibody and antigen at single antigenic sites. Within each antigenic site, the variable region of the antibody “arm” interacts through weak non-covalent forces with antigen at numerous sites; the more interactions, the stronger the affinity.

“Avidity” is informative measure of the overall stability or strength of the antibody-antigen complex. It is controlled by three major factors: antibody epitope affinity; the valency of both the antigen and antibody; and the structural arrangement of the interacting parts. Ultimately these factors define the specificity of the antibody, that is, the likelihood that the particular antibody is binding to a precise antigen epitope.

“Amino acid” means both naturally occurring amino acids and synthetic amino acids, as well as amino acid analogs and amino acid mimetics that function in a manner similar to the naturally occurring amino acids.

“Binding specificity” is the ability of an individual antibody combining site to react with only one antigenic determinant. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody. “Specific binding” between two entities is denoted by an equilibrium constant (K_{A}). The phrase “specifically (or selectively) binds” to an antibody (e.g., a C5-binding antibody) refers to a binding reaction that is determinative of the presence of a cognate antigen (e.g., a human C5 or cynomolgus C5) in a heterogeneous population of proteins and other biologics. In addition to the equilibrium constant (K_{A}) noted above, a C5-binding antibody typically binds to C5 with an affinity that is at least two-fold greater than its affinity for binding to a non-specific antigen (e.g., complement C3, complement C4, blood serum albumin (BSA)). The phrases “an antibody recognizing an antigen” and “an antibody specific for an antigen” are used interchangeably herein with the term “an antibody which binds specifically to an antigen”.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

“Complement C5 protein” or “C5” are used interchangeably and refer to the C5 protein in the complement pathway. Purified human C5 protein can be obtained from Quidel, 10165 McKellar Ct, San Diego Calif. USA (Cat. Number A403).

A “composition” is a combination of active agent (such as an anti-C5 antibody) and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives, for example; proteins, peptides, amino acids, lipids, and carbohydrates (e.g., sugars, including monosaccharides and oligosaccharides; derivatized sugars such as alditols, aldonic acids, esterified sugars and the like; and polysaccharides or sugar polymers), which can be present singly or in combination, comprising alone or in combination 1-99.99% by weight or volume. Carbohydrate excipients include, for example; monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. A carrier can also include includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-quadrature-cyclodextrin), polyethylene glycols, flavoring agents, antimicrobial agents, sweeteners, antioxidants, antistatic agents, surfactants (e.g., polysorbates such as TWEEN 20™ and TWEEN 80™), lipids (e.g., phospholipids, fatty acids), steroids (e.g., cholesterol), and chelating agents (e.g., EDTA).

The terms “cross-block”, “cross-blocked” and “cross-blocking” are used interchangeably herein to mean the ability of an antibody or other binding agent to interfere with the binding of other antibodies or binding agents to C5 in a standard competitive binding assay. The ability or extent to which an antibody or other binding agent is able to interfere with the binding of another antibody or binding molecule to C5, and therefore whether it can be said to cross-block according to the invention, can be determined using standard competition binding assays. One suitable assay involves the use of the Biacore® technology (e.g. by using the BIAcore 3000 instrument (Biacore, Uppsala, Sweden)), which can measure the extent of interactions using surface plasmon resonance technology. Another assay for measuring cross-blocking uses an ELISA-based approach.

An “effective amount” is an amount (e.g., of an anti-C5 antibody) sufficient to effect beneficial or desired results. An effective amount can be administered in one or more administrations, applications or dosages.

“Epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The “fovea” or “fovea centralis” is a part of the eye located in the center most part of the macula in the retina. The fovea is responsible for any activity where visual detail is necessary, for example, reading text or driving.

“Fundus” of the eye refers to the interior surface of the eye opposite the lens. The fundus includes the fovea, retina, optic disc and macula.

A “gene” is a coding region operably linked to appropriate regulatory sequences capable of regulating the expression of the polypeptide in some manner. A gene includes untranslated regulatory regions of DNA (e.g., promoters, enhancers, repressors, etc.) preceding (upstream) and following (downstream) the coding region (open reading frame, ORF) as well as, where applicable, intervening sequences (i.e., introns) between individual coding regions (i.e., exons). Genes may also include sequences located on both the 5′- and 3′-end of the sequences, which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers, which control or influence the transcription of the gene. The 3′-flanking region may contain sequences, which direct the termination of transcription, posttranscriptional cleavage and polyadenylation.

“Gene expression” describes the expression of genes as mRNA or protein. A “gene product” refers to the nucleic acids or amino acids (e.g., peptide or polypeptide) generated when a gene is transcribed and translated.

The terms “geographic atrophy” or “GA” refer to a medical condition characterized by loss of choriocapillaris and the overlying retinal pigment epitheium (RPE) of the eye (Scholl et al. (2009) PLos One 4(10) e7418). Vision loss occurs when the degeneration of the RPE cells leads to the loss of photoreceptor cells, both rods and cones, in the macula (the area of best vision). A “scotoma” refers to the blind spot in a subject's vision caused by the loss of these cells.

The term “haplotype” in the context of this disclosure refers to a group of SNPs that do not appear to recombine independently and that can be grouped together in blocks of SNPs. Hence, SNP's that constitute a haplotype are in linkage disequilibrium and thus tend to be inherited together. “Haplotype” also refers to the particular combinations of polymorphic variants observed in a population at polymorphic sites on a single chromosome or within a region of a single chromosome. A “haplotype,” as described herein, refers to any combination of SNPs or polymorphic sites. A haplotype can comprise two or more SNPs and the length of a genome region comprising a haplotype may vary from few hundred bases up to hundreds of kilo bases. It is recognized by those skilled in the art that the same haplotype can be described differently by determining the haplotype defining alleles from different nucleic acid strands. SNP's described herein are differentially present in human individuals and their specific sequence is indicative for the responsiveness to anti-C5 antibody treatment. Therefore, these SNPs and the haplotypes comprising said SNPs have diagnostic value for risk assessment and treatment efficacy in an individual. Detection of SNPs or polymorphic regions forming haplotypes can be accomplished by methods known in the art used for detecting nucleotides at polymorphic sites (see also definition of Linkage Disequilibrium below).

The term “human antibody” is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). Human monoclonal antibodies can be produced by a hybridoma which includes a B cell obtained from a transgenic nonhuman animal, e.g., a transgenic mouse, having a genome comprising a human heavy chain transgene and a light chain transgene fused to an immortalized cell.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). (See, e.g., Morrison et al. (1984) Proc. Natl. Acad. Sci. U.S.A. 81:6851-6855, Morrison and Oi (1988) Adv. Immunol. 44:65-92, Verhoeyen et al. (1988) Science 239:1534-1536, Padlan (1991) Molec. Immun. 28:489-498, and Padlan (1994) Molec. Immun. 31:169-217, 1994). Other examples of human engineering technology include the technology disclosed in U.S. Pat. No. 5,766,886.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same over a specified region, when compared and aligned for maximum correspondence over a comparison window, or designated region as measured using one of the following sequence comparison algorithms or by manual alignment and visual inspection. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A “comparison window” includes reference to a segment of any one of the number of contiguous positions. Methods of alignment of sequences for comparison are well known in the art. Optimal alignment of sequences for comparison can be conducted, e.g., by the local homology algorithm of Smith and Waterman ((1970) Adv. Appl. Math. 2:482c), or by the homology alignment algorithm of Needleman and Wunsch ((1970) J. Mol. Biol. 48:443), by the search for similarity method of Pearson and Lipman ((1988) Proc. Natl. Acad. Sci. U.S.A. 85:2444), by computerized implementations of these algorithms (GAP, BESTFIT, FASTA, and TFASTA in the Wisconsin Genetics Software Package, (Genetics Computer Group, 575 Science Dr., Madison Wis. USA), or by manual alignment and visual inspection, as shown e.g. by Brent et al. ((2003) Current Protocols in Molecular Biology, John Wiley & Sons, Inc., Ringbou, ed.). Two examples of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410 respectively. Software for performing BLAST analyses is publicly available through the National Center for Biotechnology Information (NCBI). The BLAST algorithm also performs a statistical analysis of the similarity between two sequences (see, e.g., Karlin and Altschul (1993) Proc. Natl. Acad. Sci. U.S.A. 90:5873-5787). The percent identity between two amino acid sequences can also be determined using the algorithm of E. Meyers and W. Miller (1988) Comput. Appl. Biosci. 4:11-17, which has been incorporated into the ALIGN program (version 2.0). In addition, the percent identity between two amino acid sequences can be determined using the Needleman and Wunsch ((1970) J. Mol. Biol. 48:444-453) algorithm which has been incorporated into the GAP program in the GCG software package (available from the National Institutes of Health). Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “isolated” means separated from constituents, cellular and otherwise, in which the polynucleotide, peptide, polypeptide, protein, antibody or fragment(s) thereof, are normally associated with in nature. For example, an isolated polynucleotide is separated from the 3′ and 5′ contiguous nucleotides with which it is normally associated within its native or natural environment, e.g., on the chromosome. A “concentrated,” “separated” or “diluted” polynucleotide, peptide, polypeptide, protein, antibody or fragment(s) thereof, is distinguishable from its naturally occurring counterpart in that the concentration or number of molecules per volume is greater in a “concentrated” version or less than in a “separated” version than that of its naturally occurring counterpart. A polynucleotide, peptide, polypeptide, protein, antibody, or fragment(s) thereof, which differs from the naturally occurring counterpart in its primary sequence or, for example, by its glycosylation pattern, need not be present in its isolated form since it is distinguishable from its naturally occurring counterpart by its primary sequence or, alternatively, by another characteristic such as glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

The term “isolated antibody” refers to an antibody that is substantially free of other antibodies having different antigenic specificities (e.g., an isolated antibody that specifically binds C5 is substantially free of antibodies that specifically bind antigens other than C5). An isolated antibody that specifically binds C5 may, however, have cross-reactivity to other antigens. Moreover, an isolated antibody may be substantially free of other cellular material and/or chemicals.

The term “isotype” refers to the antibody class (e.g., IgM, IgE and IgG such as IgG1 or IgG4) that is provided by the heavy chain constant region genes. Isotype also includes modified versions of one of these classes, where modifications have been made to alter the Fc function, for example, to enhance or reduce effector functions or binding to Fc receptors.

The term “K_{assoc}” or “Ka” refers to the association rate of a particular antibody-antigen interaction, whereas the term “K_{dis}” or “Kd,” refers to the dissociation rate of a particular antibody-antigen interaction. The term “K_{D}” refers to the dissociation constant, which is obtained from the ratio of Kd to Ka (i.e., Kd/Ka) and is expressed as a molar concentration (M). K_{D} values for antibodies can be determined using methods well established in the art. A method for determining the K_{D} of an antibody is by using surface plasmon resonance, or using a biosensor system such as a BIAcore® system (GE Healthcare, Piscataway, USA).

“Linkage Disequilibrium” or “LD” refers to a situation in which two or more allelic variants are linked, i.e., there is a non-random correlation between allelic variants at two or more polymorphic sites in individuals in a population. LD is commonly denoted by a capital D. Normalizing D by dividing it by the theoretical maximum for the observed allele frequencies results in D′. A value of 0 for D′ indicates that the examined loci are in fact independent of one another, while a value of 1 demonstrates complete dependency. Two or more allelic variants/SNPs that are linked are said to be in linkage disequilibrium. In general, allelic variants that are part of a haplotype or haplotype block are in linkage disequilibrium. A variety of methods/metrics are known in the art to evaluate the extent to which any two polymorphic variants (alleles) or SNPs are in LD. Suitable metrics include D′, r2, and others (see, e.g., Hedrick P W et al. (1987) Genetics 117(2):331-41). Polymorphic variants or SNPs are in “strong LD”, and forming a haplotype if D′>0.8.

A “marker” or “biomarker” is a nucleic acid or polypeptide and the presence or absence of a risk allele or differential expression of the polypeptide is used herein to determine the presence of GA, the progression of GA, sensitivity to an anti-C5 antibody, or a combination thereof.

A “monoclonal antibody” or “monoclonal antibody composition” refers to a preparation of antibody molecules of single molecular composition. A monoclonal antibody composition displays a single binding specificity and affinity for a particular epitope.

A “mutant,” or “mutation” is any change in DNA or protein sequence that deviates from wild type. This includes without limitation; single base nucleic acid changes or single amino acid changes, insertions, deletions and truncations of the gene and its corresponding protein.

A “nucleic acid” or “polynucleotide” refers a polymeric form of nucleotides of any length, either deoxyribonucleotides or ribonucleotides or analogs thereof, in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; and Rossolini et al. (1994) Mol. Cell. Probes 8:91-98).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “pharmaceutically acceptable carrier” includes any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers that are acceptable for use in vivo. For examples of carriers, stabilizers and adjuvants, see Remington's Pharmaceutical Science, 15th Ed. (1975) Mack Publ. Co., Easton and the Physician's Desk Reference, 52nd ed. (1998) Medical Economics, Montvale N.J.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson M et. al. (1991) PCR: A Practical Approach, IRL Press at Oxford University Press. All processes of producing replicate copies of a polynucleotide, such as PCR or gene cloning, are collectively referred to herein as “replication.” A primer can also be used as a probe in hybridization reactions, such as Southern or Northern blot analyses (see, Sambrook et al. (1989) Molecular Cloning: A Laboratory Manual, 2nd edition).

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

The term “recombinant human antibody” includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell.”

The terms “solid phase support” and “solid support” are used interchangeably and are not limited to a specific type of support. Rather a large number of supports are available and are known to one of ordinary skill in the art. Solid phase supports include silica gels, resins, derivatized plastic films, glass beads, plastic beads, alumina gels, microarrays, and chips. “Solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc./Peninsula Laboratories San Carlos, Calif.), polyHIPE(R)™ resin (obtained from Aminotech, Canada), polyamide resin (obtained from Bachem/Peninsula Laboratories, San Carlos, Calif. USA), polystyrene resin grafted with polyethylene glycol (TentaGelR™, Rapp Polymere, Tubingen, Germany), or polydimethylacrylamide resin (obtained from Milligen/Biosearch, CA USA). A polynucleotide also can be attached to a solid support for use in high throughput screening assays. Intl. Pat. Appl. No. WO 97/10365, for example, discloses the construction of high density oligonucleotide chips. See also, U.S. Pat. Nos. 5,405,783; 5,412,087 and 5,445,934. As an example, transcriptional activity can be assessed by measuring levels of messenger RNA using a gene chip such as the Affymetrix® HG-U133-Plus-2 GeneChips (Affymetrix, Santa Clara, Calif. USA). High-throughput, real-time quanititation of RNA of a large number of genes of interest thus becomes possible in a reproducible system.

The term “SNP” refers to a “single nucleotide polymorphism”. A “SNP” is a genetic variation between individuals; e.g., a single base position in the DNA of organisms that is variable. A SNP defines a specific allele of a given gene and can define a “risk allele” as described above. “SNPs” is the plural of SNP. A SNP occurs at a polymorphic site occupied by a single nucleotide, which is the site of variation between allelic sequences. The site is usually preceded by and followed by highly conserved sequences of the allele. SNPs are most frequently diallelic. A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference. A SNP may also refer to a polymorphic site on a single chromosome or within a region of a single chromosome, wherein the SNP might refer to an insertion or deletion of several base pairs. Hence, the term “SNP” refers also to a region of a gene having one of several nucleotide sequences found in that region of the gene in different individuals in a population.

The term “stringent hybridization conditions” refers to conditions under which a nucleic acid probe will specifically hybridize to its target subsequence, and to no other sequences. The conditions determining the stringency of hybridization include: temperature, ionic strength, and the concentration of denaturing agents such as formamide. Varying one of these factors may influence another factor and one of skill in the art will appreciate changes in the conditions to maintain the desired level of stringency. An example of a highly stringent hybridization is: 0.015M sodium chloride, 0.0015M sodium citrate at 65-68° C. or 0.015M sodium chloride, 0.0015M sodium citrate, and 50% formamide at 42° C. (Sambrook et al. (1989) Molecular Cloning: A Laboratory Manual, 2nd edition). An example of a “moderately stringent” hybridization is the conditions of: 0.015M sodium chloride, 0.0015M sodium citrate at 50-65° C. or 0.015M sodium chloride, 0.0015M sodium citrate, and 20% formamide at 37-50° C. The moderately stringent conditions are used when a moderate amount of nucleic acid mismatch is desired. When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

The terms “subject,” “individual” or “patient” are used interchangeably herein, which refers to a vertebrate, preferably a mammal, more preferably a human.

Mammals include, but are not limited to, mice, simians, humans, farm animals, sport animals, and pets.

The term “therapeutically effective amount” in the context of administering an therapeutically effective amount as used herein typically refers to an amount of an active ingredient (e.g. anti-C5 antibody) which, when administered to a subject, is sufficient to provide a therapeutic benefit.

The term “treating” includes the administration of compositions or antibodies to prevent or delay the onset of the symptoms, complications, or biochemical indicia of a disease (e.g., GA), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

The term “vector” refers to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms “plasmid” and “vector” are often used interchangeably because plasmids are the most commonly used forms of vectors. However, the term “vector” includes such other forms of expression vectors, such as viral vectors (e.g., replication defective retroviruses, adenoviruses and adeno-associated viruses), which serve equivalent functions.

### Geographic Atrophy

The progression of GA has been well characterized in a number of studies (Sunness et al. (2007) Ophthalmol. 114(2):271-7; Bindewald et al. (2005) Invest. Ophthalmol. Vis. Sci. 46(9):3309-14; Holz et al. (2007) Am. J. Ophthalmol. 143(3):463-72). For example, the Age Related Eye Disease Study (AREDS), where 3640 participants with early GA were randomized to receive treatment with placebo, zinc or antioxidants or both and were followed for a mean of 6 years (Lindblad et al. (2009) Arch. Ophthalmol. 127(9):1168-74). Zinc and antioxidants had previously been shown to decrease the risk for the development of wet AMD, but these were shown in the AREDS to have no effect on the development of GA.

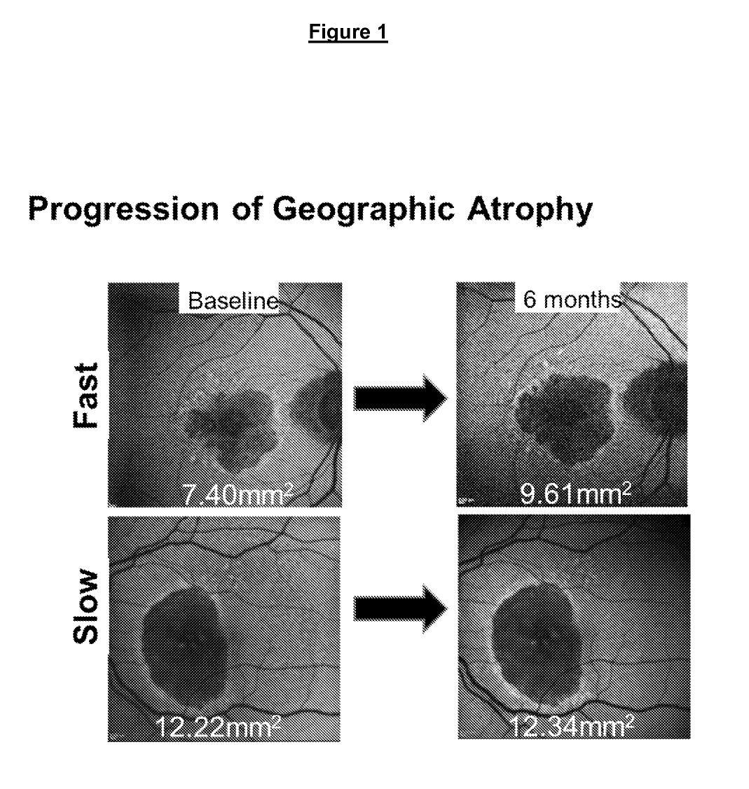

Because most eyes with GA maintain normal visual acuity until the atrophic lesions expand into the fovea, disease progression in all of these studies has been characterized by the growth of the atrophic lesions. In studies where the mean length of follow-up ranged between 1.5 to 6 years, lesion growth was linear over time with mean annualized growth rates ranging between 1.3 and 2.8 mm²/year. The approximate 2-fold difference in annualized lesion growth rates between studies is likely due to differences in mean lesion area at baseline. The AREDS (Lindblad et al. (2009, supra) and Sunness et al. (2007, supra) have shown that baseline lesion area is an important predictor of subsequent lesion growth with large lesions growing faster than smaller lesions, and with a linearity of lesion growth over time. For an example of slow and fast GA lesion growth, see FIG. 1.

In the AREDS, the length of time between the first diagnosis of geographic atrophy and lesion progression into the fovea was 2.5 years. Over this time course, best corrected visual acuity decreased, and the subject's ability to read print was reduced by an average of 3.7 letters. Five years later as the lesions expanded into the fovea, the mean vision loss was 22 letters. These results are consistent with those of Sunness et al. (1999) Ophthalmol. 106(9):1768-79 and those of Schatz and McDonald (1989) Ophthalmol. (10):1541-51. Both groups have reported that among GA patients who entered the studies with minimal impairment of visual function, 7% to 15% lost 15 or more letters over the subsequent 12 months.

This pattern of vision loss in GA is markedly different from that of that of wet AMD, where vision loss is not only more rapid, but at least partially reversible. In studies with verteporfin, the first agent approved as a treatment of wet AMD, 54% of patients lost 15 or more letters within 12 months when left untreated (Bressler et al. (2000) BMJ 321(7274): 1425-1427; Soubrane et al. (2001) Br. J. Ophtha. 85(4):483-495). In studies with ranibizumab, after 12 months of treatment 30 to 40% of wet AMD patients recovered 15 or more letters of visual acuity (Rosenfeld et al. (2006) N. Engl. J. Med. 355(14):1419-31; Brown et al. (2006) N. Engl. J. Med. 355(14):1432-44.

### Assessment of Geographic Atrophy in a Subject

Based on images of the fundus, GA is defined as any sharply demarcated area of apparent absence of retinal pigmented epithelial cells (RPE), larger than 175 μm, with visible choroidal vessels and no neovascularization. This definition is based on histology studies that have characterized clinically visible areas of atrophy as areas of cell death in the retinal pigmented epithelium and outer neurosensory layers; with the loss of these cell layers, the underlying choriocapillaris becomes visible. Because retinal pigmented epithelial cells and neurosensory cells do not replicate, the loss of these cells results in irreversible retinal damage and loss of visual function. Typically the atrophic lesions initially appear in the extrafoveal region where they expand, coalesce and eventually progress into the center of the fovea, but only late in the course of the disease (Schatz and McDonald (1989) Ophthalmol. 96: 1541-1551; Sunness et al. (1997) Ophthalmol. 104: 1677-1691). Central vision loss begins when atrophic lesions develop in the parafoveal region; severe vision loss occurs when these areas of atrophy expand into the foveola. If the center of the fovea is not immediately affected, good central vision is maintained until late in the disease (Schatz and McDonald (1989) Ophthalmol. 96: 1541-1551). When the center of the fovea is not fully involved, atrophic lesions impair visual performance by limiting the size of the functioning fovea so that when reading, only a portion of a word is visually acute (Sunness et al. (1997) Ophthalmol. 104: 1677-1691; Sunness et al. (1999) Ophthalmol. 106(5):910-9). As described by Sunness (1997, supra), patients with GA sometime report a paradox of being able to read newsprint, but not larger print such as news headlines; this apparent contradiction is considered a consequence of the headline letters being too large to fit into the area of the functioning fovea.

Using the expansion of atrophic GA lesions as a measure of disease progression is appropriate because the lesions represent irreversible retinal damage. Moderate to severe vision loss occurs only late in the disease when the atrophic GA lesions progress into the center of the fovea. If retinal function measures were to be used to assess disease progression, disease progression would not be evident until late in the course of the disease at which time an intervention treatment would have little benefit because of the extent of irreversible retina damage.

### Assay for Biomarkers of Geographic Atrophy

The invention provides methods for predicting therapeutic responsiveness of a subject, e.g. a human subject, to anti-C5 antibody treatment, based on the presence or absence of particular genetic markers in the subject to be treated. Such an ability to assess the likelihood that treatment will or will not be clinically effective is typically done before treatment with the anti-C5 antibody is begun in the subject. However, an ability to assess the likelihood that treatment will or will not be clinically effective can be exercised after treatment has begun, but before an indicator of clinical effectiveness (e.g. the slowing of GA) is also useful in clinical management of the patient.

Characterization of at least two risk alleles of a subject at the ARMS2 or CFH loci can be done by using any of the techniques well known in the art. For example, any of the regions of the individual risk alleles may be sequenced. Any of the well-known methods for sequencing one or both strands of DNA may be used in the methods of the invention, such as the methods described in, for example, U.S. Pat. No. 5,075,216, Engelke et al. (1988) Proc. Natl. Acad. Sci. U.S.A. 85, 544-548 and Wong et al. (1987) Nature 330, 384-386; Maxim and Gilbert (1977) Proc. Natl. Acad. Sci. U.S.A. 74:560; or Sanger (1977) Proc. Natl. Acad. Sci. U.S.A. 74:5463. In addition, any of a variety of automated sequencing procedures can be used. See, e.g., Naeve et al. (1995) Biotechniques 19:448. Sequencing by mass spectrometry can be used. See, e.g., Intl. Pat. Appl. No. WO 94/16101; Cohen et al. (1996) Adv. Chromatogr. 36:127-162; and Griffin et al. (1993) Appl. Biochem. Biotechnol. 38:147-159.

Determining the presence or absence of a risk allele in a biological sample may be accomplished using any well-known technique such as polymerase chain reaction (PCR) amplification reaction, reverse-transcriptase PCR analysis, single-strand conformation polymorphism analysis (SSCP), mismatch cleavage detection, heteroduplex analysis, Southern blot analysis, Western blot analysis, deoxyribonucleic acid sequencing, restriction fragment length polymorphism analysis, haplotype analysis, serotyping, and combinations or sub-combinations thereof. In particular, the risk allele assay can be by sequence-specific primer (SSP) typing, sequence-specific oligonucleotide (SSO) typing, sequence based typing (SBT), DNA amplification such as polymerase chain reaction (PCR) or microarray analysis.

For example, a mRNA sample may be obtained from the subject and expression of mRNA(s) encoded by the risk allele in the mRNA sample may be detected using standard molecular biology techniques, such as PCR analysis. A preferred method of PCR analysis is reverse transcriptase-polymerase chain reaction (RT-PCR). Other suitable systems for mRNA sample analysis include microarray analysis (e.g., using Affymetrix's microarray system or Illumina's BeadArray Technology).

Determining the presence or absence of a risk allele of the ARMS2 or CFH gene may include restriction fragment length polymorphism analysis. Restriction fragment length polymorphism analysis (RFLPS) is based on changes at a restriction enzyme site. Moreover, the use of sequence specific ribozymes (see, for example, U.S. Pat. No. 5,498,531) may be used to score for the presence of a specific ribozyme cleavage site.

Another technique for determining the presence or absence of a risk allele of ARMS2 or CFH involves hybridizing DNA segments which are being analyzed (target DNA) with a complimentary, labeled oligonucleotide probe as described by Wallace et al. (1981) Nucl. Acids Res. 9, 879-894. Since DNA duplexes containing even a single base pair mismatch exhibit high thermal instability, the differential melting temperature may be used to distinguish target DNAs that are perfectly complimentary to the probe from target DNAs that only differ by a single nucleotide. This method has been adapted to detect the presence or absence of a specific restriction site, as described in, for example, U.S. Pat. No. 4,683,194. The method involves using an end-labeled oligonucleotide probe spanning a restriction site which is hybridized to a target DNA. The hybridized duplex of DNA is then incubated with the restriction enzyme appropriate for that site. Reformed restriction sites will be cleaved by digestion in the pair of duplexes between the probe and target by using the restriction endonuclease. The specific restriction site is present in the target DNA if shortened probe molecules are detected.

Other methods for determining the presence or absence of a risk allele of ARMS2 or CFH include methods in which protection from cleavage agents is used to detect mismatched bases in RNA/RNA or RNA/DNA heteroduplexes, as described for example by Myers et al. (1985) Science 230:1242. In general, the technique of “mismatch cleavage” starts by providing heteroduplexes formed by hybridizing (labeled) RNA or DNA containing the polymorphic sequence with potentially polymorphic RNA or DNA obtained from a sample. The double-stranded duplexes are treated with an agent which cleaves single-stranded regions of the duplex such as which will exist due to base-pair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels. See, for example, Cotton et al. (1988) Proc. Natl. Acad. Sci. U.S.A. 85:4397; Saleeba et al. (1992) Methods Enzymol. 217:286-295. In a preferred embodiment, the control DNA or RNA can be labeled for detection.

In another embodiment, alterations in electrophoretic mobility may be used to determine the presence or absence of a risk allele of ARMS2 or CFH. For example, single strand conformation polymorphism (SSCP) may be used to detect differences in electrophoretic mobility between various indicative risk alleles of ARMS2 or CFH, as described for example by: Orita et al. (1989) Proc. Natl. Acad. Sci. U.S.A. 86:276; Cotton (1993) Mutat. Res. 285:125-144; and Hayashi (1992) Genet. Anal. Tech. Appl. 9:73-79. Single-stranded DNA fragments of sample and control nucleic acids can be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

The movement of a nucleic acid molecule in polyacrylamide gels containing a gradient of denaturant (denaturing gradient gel electrophoresis (DGGE)) can be used to assess the presence or absence of a risk allele (Myers et al. (1985) Nature 313:495). When DGGE is used as the method of analysis, DNA can be modified to ensure that it does not completely denature, for example by adding a GC clamp of approximately 40 base pairs (bp) of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for determining the presence or absence of risk alleles of the ARMS2 or CFH gene include, but are not limited to, selective oligonucleotide hybridization, selective amplification, or selective primer extension. For example, oligonucleotide primers may be prepared in which the polymorphic region is placed centrally and then hybridized to target DNA under conditions which permit hybridization only if a perfect match is found (Saiki et al. (1986) Nature 324:163; Saiki et al. (1989) Proc. Natl. Acad. Sci. U.S.A. 86:6230). Such risk allele specific oligonucleotides are hybridized to PCR amplified target DNA or a number of different polymorphisms when the oligonucleotides are attached to the hybridizing membrane and hybridized with labeled target DNA.

Another process for determining the presence or absence of risk alleles of ARMS2 or CFH is the primer extension process which consists of hybridizing a labeled oligonucleotide primer to a template RNA or DNA and then using a DNA polymerase and deoxynucleoside triphosphates to extend the primer to the 5′ end of the template. Resolution of the labeled primer extension product is then done by fractionating on the basis of size, e.g., by electrophoresis via a denaturing polyacrylamide gel. This process is often used to compare homologous DNA segments and to detect differences due to nucleotide insertion or deletion. Differences due to nucleotide substitution are not detected since size is the sole criterion used to characterize the primer extension product. Additional well known methods for risk allele genotyping are: (1) Dynamic allele-specific hybridization (DASH) genotyping, described by (Howell W et al. (1999) Nat. Biotechnol. 17(1):87-8); (2) SNP detection through molecular beacons, described by (Abravaya et al. (2003) Clin. Chem. Lab. Med. 41:468-474); (3) High-density oligonucleotide SNP microarrays, described by (Rapley R and Harbron S (Eds.) (2004) Molecular Analysis and Genome Discovery. Chichester. John Wiley & Sons Ltd.); and (4). Flap endonucleases, for example the Invader assay for SNP genotyping (Olivier (2005) Mutat. Res. 573(1-2):103-10).

In the event that a risk allele is located in the promoter region, or another non-coding region having influence on the expression rate of the gene with the risk allele, the mRNA or protein levels might be affected. In such a situation, the presence of the risk allele cannot be determined on the basis of the mRNA or protein sequence of said respective gene. However presence of the risk allele could be determined indirectly by mRNA or protein levels measurements. Hence, in another embodiment of the invention, the herein disclosed methods can comprise an additional or alternative step of determining the mRNA or protein level of a certain gene or gene product as an indirect determination method for the presence of a risk allele in ARMS2 or CFH.

Haplotype analysis of one or more polymorphic sites around risk alleles of ARMS2 or CFH may also be used for determining the presence or absence of additional indicative risk alleles and may include, for example, use of family pedigrees, molecular techniques and/or statistical inference.

The biological sample of the subject to be tested can be blood, blood-derived product (such as buffy coat, serum, and plasma), lymph, urine, tear, saliva, cerebrospinal fluid, buccal swabs, sputum, hair roots, leukocyte sample or tissue samples or any combination thereof.

The reagent, agent or device with which the biological sample is contacted may be, for example, a PCR/sequencing primer(s), nucleotides and enzymes suitable for amplifying and/or sequencing and/or labeling the ARMS2 or CFH risk alleles or an antibody capable of detecting one of the risk alleles a restriction enzyme, and/or a microarray.

### Detection of Risk Alleles

The risk alleles that can be tested on the presence or absence of ARMS2 SNP rs10490924 (SEQ ID NO. 1) or CFH SNP rs1061170 (SEQ ID NO. 3) as disclosed in TABLE3 and the sequence listing. The ARMS2 SNP rs10490924 (SEQ ID NO. 1) is a G>T change with a base pair length of 1 located at position 124214448 of the human chromosome 10. The SNP rs1061170 (SEQ ID NO. 3) is a T>C change with a base pair length of 1 located at position 196659237 of the human chromosome 1. Both of these changes are shown in Table 3, with the base change bolded and underlined.

Because some SNPs or polymorphic regions exist in the human ARMS2, CFH genes and in the nearby genomic regions that are in same LD with the SNP rs10490924 or the SNP rs1061170. SNPs or polymorphic regions that are in same LD with the SNP rs10490924 or the SNP rs1061170 will be equally suited to be used as markers for predicting therapeutic responsiveness to an anti-C5 antibody treatment. Therefore, detection of SNP rs11200638 that is located in the promoter region of HTRA1 (SEQ ID NO. 2) provides the equivalent information as the detection of the SNP rs10490924 in ARMS2 (SEQ ID NO. 1) because the two SNPs are in same LD.

### The Role of Complement in GA

The normal role of complement, which is part of the innate immune system, is in host defense. The complement cascade is activated by at least three major pathways. The classical pathway is typically activated by immune-complexes, the alternative pathway can be activated by unprotected cell surfaces, and the mannose binding lectin (MBL) pathway is initiated by binding of MBL to cell surface carbohydrates (Trendelenburg (2007) Swiss Med Wkly 137:413-417). All three pathways lead to the cleavage of C5 protein by the C5 convertase. The C5 protein is expressed intracellularly as a single pro-05 peptide of 1676 amino acids that consist of an 18 residue signal sequence and an Arg-rich linker sequence (RPRR) situated between the mature N-terminal β chain and the C-terminal α chain. The mature C5 has a molecular weight of about 190 kDa, and consists of two polypeptide chains (α, 115 kDa and β, 75 kDa) which are connected by disulfide bonds. The C5 convertase cleaves C5 between residues 74 and 75 of the alpha chain. The result of this cleavage is release of the C5a fragment, a potent inflammatory molecule, and the C5b fragment which initiates the membrane attack complex (MAC). The complement products, once released, do not differentiate between foreign and self-targets and, if not tightly regulated, often cause extensive damage of bystander cells and tissues in clinical conditions associated with unrestricted complement activation.

The reported normal serum concentration of human C5 is about 76±24 μg/mL (Kohler and Muller-Eberhard (1967) J. Immunol. 99(6):1211-6). A comparison of human C5 concentrations between normal individuals and GA patients has not been published, but the levels of terminal complement activation products C5a and C5b-9 have been reported to be elevated in AMD patients (Scholl et al (2008) PLoS ONE 3:e2593), implying above-normal activation and proteolysis of C5.

### Anti-C5 Antibodies

Among the C5 antibodies that can be administered to subjects to inhibit the progression of geographic atrophy include the following:

In one embodiment, the anti-C5 antibody to be administered is antibody 8109, which is described in Intl. Pat. Appl. No. WO 2010/015608, “Compositions and Methods for Antibodies Targeting Complement Protein C5” and U.S. Pat. No. 8,241,628. The CDR sequences of antibody 8109 are included herein: HCDR1 sequence (SEQ ID NO 4), HCDR2 sequence (SEQ ID NO. 5), HCDR3 sequence (SEQ ID NO. 6), LCDR1 sequence (SEQ ID NO. 7), LCDR2 sequence (SEQ ID NO. 8), and LCDR3 sequence (SEQ ID NO, 9).

In another embodiment, the anti-C5 antibody to be administered is any antibody having the CDR sequences of antibody 8109, as described in SEQ ID NOs. 4-9.

In another embodiment, the anti-C5 antibody to be administered is the humanized monoclonal antibody eculizumab (Soliris™) and other antibodies that bind to the alpha chain of human C5 are described in Intl. Pat. Appl. No. WO 95/29697 to Alexion Pharmaceuticals.

In another embodiment, the anti-C5 antibody to be administered specifically binds to the same epitope as antibody 8109.

Additional antibodies can therefore be identified based on their ability to cross-compete (e.g., to competitively inhibit the binding of, in a statistically significant manner) with the other antibodies disclosed herein in C5 binding assays. The ability of a test antibody to inhibit the binding of antibodies of the present invention to a C5 protein (e.g., human and/or cynomolgus C5) demonstrates that the test antibody can compete with that antibody for binding to C5; such an antibody may, according to non-limiting theory, bind to the same or a related (e.g., a structurally similar or spatially proximal) epitope on the C5 protein as the antibody with which it competes. In a certain embodiment, the antibody that binds to the same epitope on C5 as the antibodies of the present invention is a human monoclonal antibody. Such human monoclonal antibodies can be prepared and isolated as described herein.

C5 antibodies can be characterized by various functional assays. For example, they can be characterized by their ability to inhibit red blood cell lysis in hemolytic assays, their affinity to a C5 protein (e.g., human and/or cynomolgus C5), the epitope binning, their resistance to proteolysis, and their ability to block the complement cascade, for example, their ability to inhibit MAC formation.

Various methods can be used to measure presence of complement pathway molecules and activation of the complement system (U.S. Pat. No. 6,087,120; and Newell et al. (1982) J. Lab. Clin. Med. 100:437-44). For example, the complement activity can be monitored by (i) measurement of inhibition of complement-mediated lysis of red blood cells (hemolysis); (ii) measurement of ability to inhibit cleavage of C3 or C5; and (iii) inhibition of alternative pathway mediated hemolysis.

The two most commonly used techniques are hemolytic assays (Baatrup et al. (1992) Ann. Rheum. Dis. 51:892-7) and immunological assays (Auda et al. (1990) Rheumatol. Int. 10:185-9). The hemolytic techniques measure the functional capacity of the entire sequence-either the classical or alternative pathway. Immunological techniques measure the protein concentration of a specific complement component or split product. Other assays that can be employed to detect complement activation or measure activities of complement components in the methods of the present invention include, e.g., T cell proliferation assay (Chain et al. (1987) J. Immunol. Methods 99:221-8), and delayed type hypersensitivity (DTH) assay (Forstrom et al. 1983, Nature 303:627-629; Halliday et al. (1982), in Assessment of Immune Status by the Leukocyte Adherence Inhibition Test, Academic, New York pp. 1-26; Koppi et al. (1982) Cell. Immunol. 66:394-406; and U.S. Pat. No. 5,843,449).

Hemolytic techniques can screen both functional integrity and deficiencies of the complement system (Dijk et al. (1980) J Immunol Methods 36: 29-39; Minh et al. (1983) Clin. Lab. Haematol. 5:23-34 and Tanaka et al. (1986) J. Immunol. 86: 161-170). To measure the functional capacity of the classical pathway, sheep red blood cells coated with hemolysin (rabbit IgG to sheep red blood cells) or chicken red blood cells that are sensitized with rabbit anti-chicken antibodies are used as target cells (sensitized cells). These antigen-antibody complexes activate the classical pathway and result in lysis of the target cells when the components are functional and present in adequate concentration. To determine the functional capacity of the alternative pathway, rabbit red blood cells are used as the target cell (see, e.g., U.S. Pat. No. 6,087,120).

To test the ability of an antibody to inhibit MAC (membrane attack complex) formation, a MAC deposition assay can be performed. Briefly, zymosan can be used to activate the alternative pathway and IgM can be used to active the classic pathway. Fabs are pre-incubated with human serum and added to plates coated with zymosan or IgM. Percentage inhibition of MAC deposition can be calculated for each sample relative to baseline (EDTA treated human serum) and positive control (human serum).

The ability of an antibody to bind to C5 can be detected by labeling the antibody of interest directly, or the antibody may be unlabeled and binding detected indirectly using various sandwich assay formats known in the art.

In some embodiments, the C5-binding antibodies of the invention block or compete with binding of a reference C5-binding antibody to a C5 polypeptide. These can be fully human C5-binding antibodies described above. They can also be other mouse, chimeric or humanized C5-binding antibodies which bind to the same epitope as the reference antibody. The capacity to block or compete with the reference antibody binding indicates that a C5-binding antibody under test binds to the same or similar epitope as that defined by the reference antibody, or to an epitope which is sufficiently proximal to the epitope bound by the reference C5-binding antibody. Such antibodies are especially likely to share the advantageous properties identified for the reference antibody. The capacity to block or compete with the reference antibody may be determined by, e.g., a competition binding assay. With a competition binding assay, the antibody under test is examined for ability to inhibit specific binding of the reference antibody to a common antigen, such as a C5 polypeptide. A test antibody competes with the reference antibody for specific binding to the antigen if an excess of the test antibody substantially inhibits binding of the reference antibody.

Known competition binding assays can be used to assess competition of a C5-binding antibody with the reference C5-binding antibody for binding to a C5 protein. These include, e.g., solid phase direct or indirect radioimmunoassay (RIA), solid phase direct or indirect enzyme immunoassay (EIA), sandwich competition assay (Stahli et al. (1983) Methods in Enzymology 9:242-253); solid phase direct biotin-avidin EIA (Kirkland et al. (1986) J. Immunol. 137:3614-3619); solid phase direct labeled assay, solid phase direct labeled sandwich assay; solid phase direct label RIA using 1-125 label (Morel et al. (1988) Molec. Immunol. 25:7-15); solid phase direct biotin-avidin EIA (Cheung et al. (1990) Virology 176:546-552); and direct labeled RIA (Moldenhauer et al. (1990) Scand. J. Immunol. 32:77-82). Typically, such an assay involves the use of purified antigen bound to a solid surface or cells bearing either of these, an unlabelled test C5-binding antibody and a labelled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

To determine if the selected C5-binding monoclonal antibodies bind to unique epitopes, each antibody can be biotinylated using commercially available reagents (e.g., reagents from Pierce, Rockford, Ill. USA). Competition studies using unlabeled monoclonal antibodies and biotinylated monoclonal antibodies can be performed using a C5 polypeptide coated-ELISA plates. Biotinylated monoclonal antibody binding can be detected with a strep-avidin-alkaline phosphatase probe. To determine the isotype of a purified C5-binding antibody, isotype ELISAs can be performed. For example, wells of microtiter plates can be coated with 1 μg/ml of anti-human IgG overnight at 4° C. After blocking with 1% BSA, the plates are reacted with 1 μg/ml or less of the monoclonal C5-binding antibody or purified isotype controls, at ambient temperature for one to two hours. The wells can then be reacted with either human IgG- or human IgM-specific alkaline phosphatase-conjugated probes. Plates are then developed and analyzed so that the isotype of the purified antibody can be determined.

To demonstrate binding of monoclonal C5-binding antibodies to live cells expressing a C5 polypeptide, flow cytometry can be used. Briefly, cell lines expressing C5 (grown under standard growth conditions) can be mixed with various concentrations of a C5-binding antibody in PBS containing 0.1% BSA and 10% fetal calf serum, and incubated at 37° C. for 1 hour. After washing, the cells are reacted with fluorescein-labeled anti-human IgG antibody under the same conditions as the primary antibody staining. The samples can be analyzed by FACScan (BD Biosciences, San Jose, USA) using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

C5-binding antibodies of the invention can be further tested for reactivity with a C5 polypeptide or antigenic fragment by Western blotting. Briefly, purified C5 polypeptides or fusion proteins, or cell extracts from cells expressing C5 can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo. USA).

### Treatment of Geographic Atrophy by Anti-C5 Antibody

Once a subject has been assayed for the presence of a risk allele and predicted to be sensitive to an anti-C5 antibody, administration of any anti-C5 antibody to a patient can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Intravitreal administration can also be carried out by direct injection to the eye. Suitable dosage formulations and methods of administering the agents may be empirically adjusted.

As used in the specification and claims, the singular forms “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood that the reagents described herein are merely exemplary and that equivalents of such are known in the art.

Those skilled in the art will recognize, or be able to ascertain using no more than routine experimentation, many equivalents to the specific embodiments in accordance with the invention described herein. The scope of the invention is not intended to be limited to the above Detailed Description, but rather is as set forth in the appended claims.

The EXAMPLES, which follow, are illustrative of specific embodiments of the invention, and various uses thereof. They are set forth for explanatory purposes only, and are not to be taken as limiting the invention.

### EXAMPLES

### Example 1

### Using FAF to Determine the Progression of GA

FIG. 1 shows the progression of GA over time. The top panel shows fast progression, showing 2.21 mm² growth over 6 months. In contrast, the bottom panel shows a slow progression of GA, with just 0.12 mm² over the same time period. This was done using fundus autofluoresence (FAF). FAF is a non-invasive method of imaging retinal disorders, especially those of the retinal pigment epithelium. Retinal pigment epithelium atrophy appears as a dark patch (FIG. 1) and can be clearly delineated and measured, and thus is a useful imaging tool for following the progression or regression of GA (Solbach et al. (1997) Retina 17:385-389).

In these experiments, a multicenter, randomized, sham-control, proof-of-concept study was performed, to test the efficacy and safety of monthly intravitreal administration of 8109 in patients with GA. The study was designed to test the effect of eighteen successive 5 mg/50 μL doses of intravitreal (IVT) 8109, administered every 4 weeks (˜monthly), on the growth of GA lesions as measured by FAF from baseline to month 12. The secondary endpoints included the assessment of GA lesion growth at 6 and 18 months using FAF, and change in best corrected visual acuity. FAF was measured using a confocal laser scanner opthalmoscope an example of which has been previously described by Holz (Holz et al. (1998) Am J Ophthalmol. 125: 227-236). Serial FAF images of the same eye were aligned via software (e.g. Region Finder, Heidelberg Engineering, Dossenheim, Germany) using the baseline image. The total size of the GA lesion was measured using the processed FAF images by automated imaging analysis software (Region Finder, Heidelberg Engineering, Dossenheim, Germany; see also Schmitz-Valckenberg et al. (2004) Invest. Ophthalmol. Vis. Sci. 45: 4470-4476.) Growth of the GA lesion in mm² was measured by subtracting baseline values obtained from the Heidelberg measurement from the subsequent measurements at 3, 6, 9, 12 and 18 months.

### Example 2

### Analysis of Risk Alleles Associated with GA

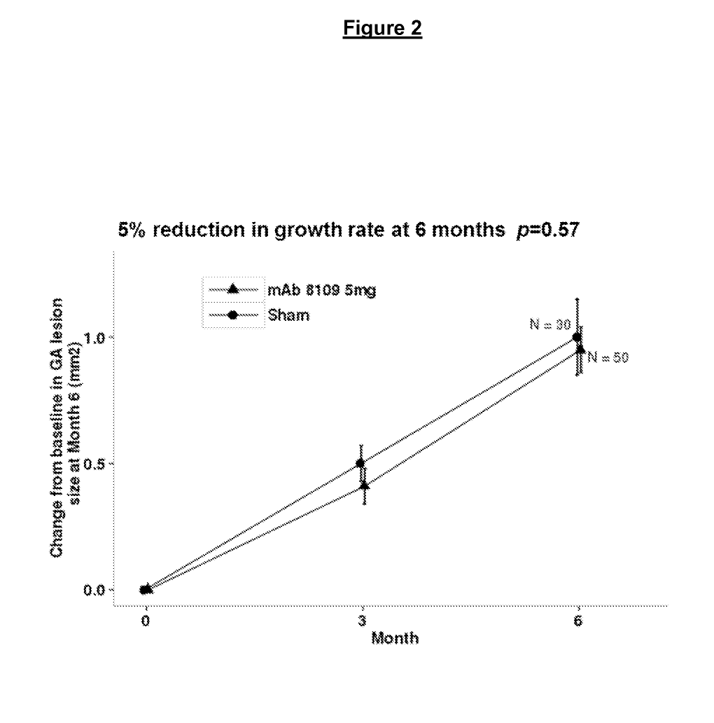

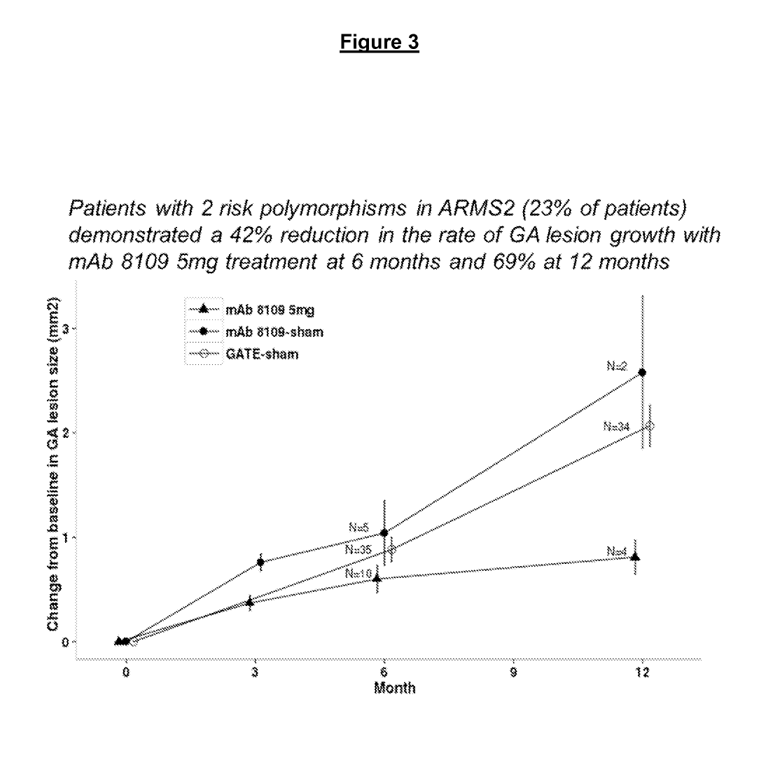

Using data from the sham arm of a study (referred to in FIG. 2 and FIG. 3 as “GATE-sham”), the genotype and clinical data from 441 patients with GA were analyzed. A significant association between the ARMS2 variant (rs10490924) and GA lesion size growth was detected. Patients who carried the ARMS2 risk allele (rs10490924) tended to progress faster than those who did not carry the risk allele (1 risk allele, p=0.022; 2 risk alleles, p=0.005). In addition, a cumulative effect of the CFH (rs1061170) and ARMS2 (rs10490924) risk alleles on GA lesion size growth was noted. Patients who carried the risk alleles tended to progress faster than those who did not carry the risk allele in the two genes (1 risk allele, p=0.007; 2 risk alleles, p=0.001; 3 risk alleles, p=0.001; 4 risk alleles, p=0.001).

There was no significant association between the CFB variant and geographic atrophy lesion size growth. The P-value of each of the risk alleles analyzed by the study were calculated, and this is provided in TABLE 1. This demonstrates that the ARMS2 and CFH risk alleles score with high statistical significance.

### Example 3

### Study to Identify a Subset of Patients that Respond to Anti-C5 Antibody Treatment

To measure the effect of anti-C5 antibody treatment of patients with GA fundus autofluorescence (FAF) was applied allowing quantitatively measurements during the treatment period.

Statistical Methods for Efficacy Analyses.

Evaluation of the drug efficacy was obtained from a statistical analysis of the change from baseline of FAF using analysis of covariance (ANCOVA) model with treatment and genotype as fixed factors. Baseline GA lesion size was included in the model as a covariate and the interaction between genotype and treatment was also evaluated in the model. The overall study population was split by the genetic marker to compare the difference of the treatment effect by anti-C5 antibody vs. sham. The difference of the treatment effect within genotype groups was evaluated using similar statistical model without interaction term.

Clinical Samples:

Genomic DNA from individuals was extracted from whole blood according to the instructions from Gentra Systems, Inc. (Minneapolis Minn. USA).

Genotyping Assay:

The DNA samples were genotyped for risk alleles in ARMS2 and CFH. Genotyping was performed using TaqMan Assays-by-Design and Assays-on-Demand (Applied Biosystems, Foster City Calif. USA) on an ABI 7900 sequencer. Genotyping used 1 ng of genomic DNA according to manufacturer's instruction.

### Example 4

### Risk Alleles Involving the Complement Pathway Indicate Sensitivity to Anti-C5 Antibodies

A combination of risk alleles in the CFH and ARMS2 were analyzed to determine if patients with this risk allele profile would be sensitive to anti-C5 antibodies. Thus, patients positive for the CFH and ARMS2 risk alleles would indicate that treatment with anti-C5 antibodies would prove effective in reducing GA lesions.

Genomic DNA was extracted from peripheral blood leukocytes according to established protocols. Genotyping was done by TaqMan SNP Genotyping. TaqMan Pre-Designed SNP Genotyping Assays (Applied Biosystems, Foster City, Calif., USA) were performed according to the manufacturer's instructions and were analyzed with a 7900HT Fast Real-Time PCR System (Applied Biosystems, Foster City Calif.). All SNPs showed high genotyping quality with an average call rate of 98.5%.

Eighty patients (30 sham-treated and 50 anti-C5-treated) were randomized to either receive monthly injection of 5 mg of 8109 intravitreally or received a sham injection. Prior to initiation of study, and at various intervals after the dosing, patients underwent full physical and ophthalmic examinations. The primary endpoint of the study was change in GA lesion growth as measured by fundus auto fluorescent imaging (FAF). The secondary end point included the measurement of best corrected visual acuity.

The statistical analyses were carried out using SAS Version 9.3 and software package R, Version 2.15.1 (Cary, N.C., USA). For the overall patient population of the study, a MMRM (mixed-effect model for repeated measurements, using PROC MIXED procedure in SAS) was used to quantify GA growth, measured by change from baseline in GA lesion size in study eyes. The model included: treatment, square root of baseline lesion size, smoking status, other baseline lesion characteristics (including unilateral/bilateral, unifocal/multifocal and foveal/extrafoveal), time as a continuous variable, interaction of time and treatment, and random intercept and slope for subjects.

Taking the patient population as a whole, patients treated with monthly injection of 8109 demonstrated only a 5% reduction in the rate of GA lesion growth as compared to sham treated patients. This is shown in FIG. 2.

However, when patients were segregated by risk alleles, patients with two ARMS2 risk alleles demonstrated a reduction in GA lesion growth rate with 8109 of 42% at month 6 and 69% at month 12 (FIG. 3). The percentage difference is that between 8109 treated and sham treated. Note that the patients with two ARMS2 risk alleles represented 23% of the patients. The patient population with a single ARMS2 risk allele was 51.5%. Patients with only one ARMS2 risk allele were much less responsive to antibody 8109. For these patients, their response was −26.5% at month 6 and -46.7% at month 12. A summary of this data is presented in TABLE 2 below.

TABLE 2

| | | | Percentage |
|---|---|---|---|
| | | | Difference |
| | 8109 | Sham | between 8109 |
| | (Mean/SD/n) | (Mean/SD/n) | and sham |
| Month 6 | | | |
| Patients with 2 risk | 0.60 (0.41) | 1.04 (0.70) | 42.3% |
| alleles | n = 10 | n = 5 | |
| Patients with 1 risk | 1.05 (0.63) | 0.83 (0.36) | −26.5% |
| alleles | n = 23 | n = 11 | |
| Patients with 0 risk | 1.02 (0.53) | 0.87 (0.54) | −17.2% |
| alleles | n = 9 | n = 8 | |
| Month 12 | | | |
| Patients with 2 risk | 0.81 (0.31) | 2.58 (1. 04) | 68.6% |
| alleles | n = 4 | n = 2 | |
| Patients with 1 risk | 1.79 (0.92) | 1.22 (0.32) | −46.7% |
| alleles | n = 9 | n = 4 | |
| Patients with 0 risk | 2.51 (1.00) | 1.89 (1.17) | −32.8% |
| alleles | n = 3 | n = 4 | |

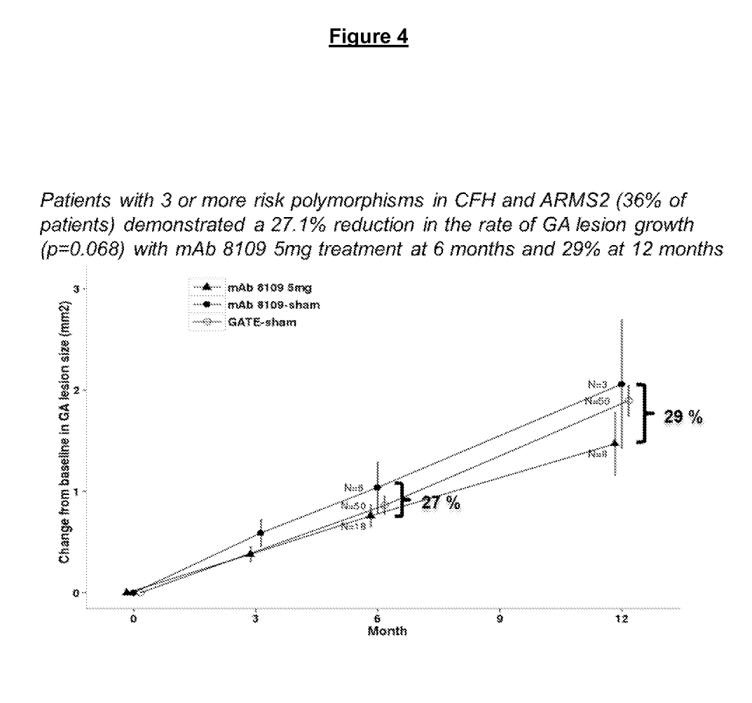

Also included in the graphs in FIG. 3 and FIG. 4 is information from the “GATE-sham” study, which is described in EXAMPLE 2.

Patients with at least three risk alleles in CFH and ARMS2 genes demonstrated a reduction in GA lesion growth rate with 8109 of 27% at month 6 and 29% at month 12, and this is shown in FIG. 4. In this group of patients, 36% contained the greater than three risk allele profile. Without being bound to any one particular hypothesis, it is possible that patients with the combination of the risk alleles in CFH and ARMS2 have a greater complement mediated pathology resulting in a more aggressive disease, but benefit more from complement inhibitors such as an anti-C5 antibody. Patients with CFH risk alleles alone did not demonstrate any response to treatment.

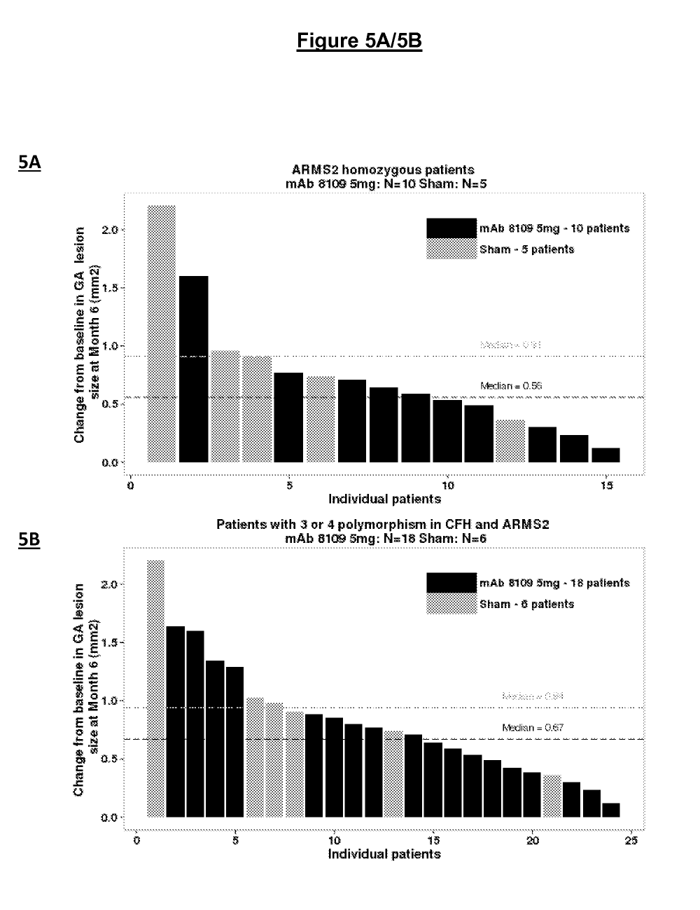

The differences in this treatment are shown graphically in the waterfall plots of FIGS. 5A and 5B. For example 5A depicts the sorted change in GA growth for the anti-C5 antibody treated patients together with sham treated patients at 6 months. This data shows a reduction in GA growth from 0.91 mm² median of sham treated to a 0.56 mm² median of anti-C5 antibody treated, a difference of 0.35 mm² over 6 months' time. This is reduction over the average reported natural GA growth rate of 2 mm² per year (Lindblad et al. (2009) Arch. Ophtha. 127(9):1168-1174).

FIG. 5B shows the sorted change in GA growth in both anti-C5 treated patients and sham treated patients, with at least 3 risk alleles in ARMS2 and CFH over sham treated.

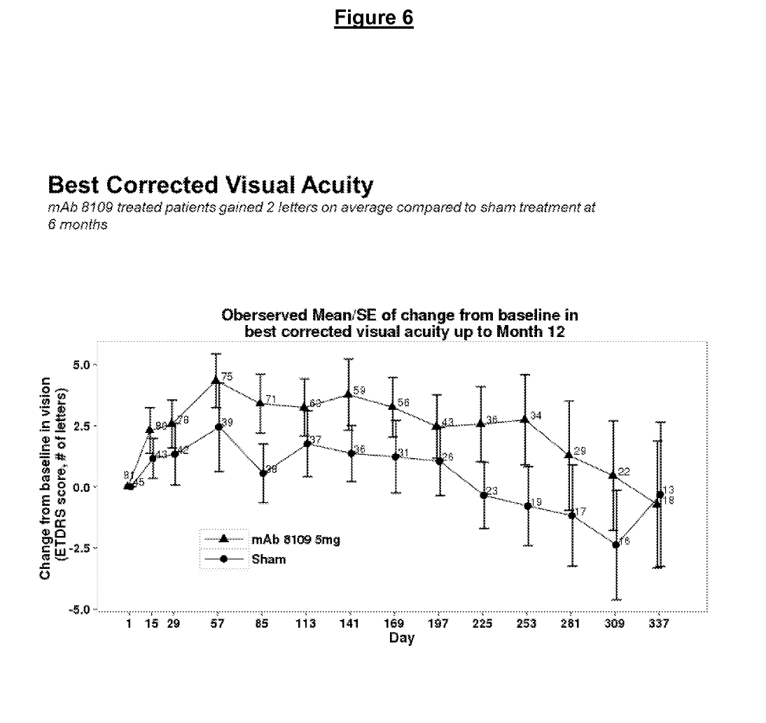

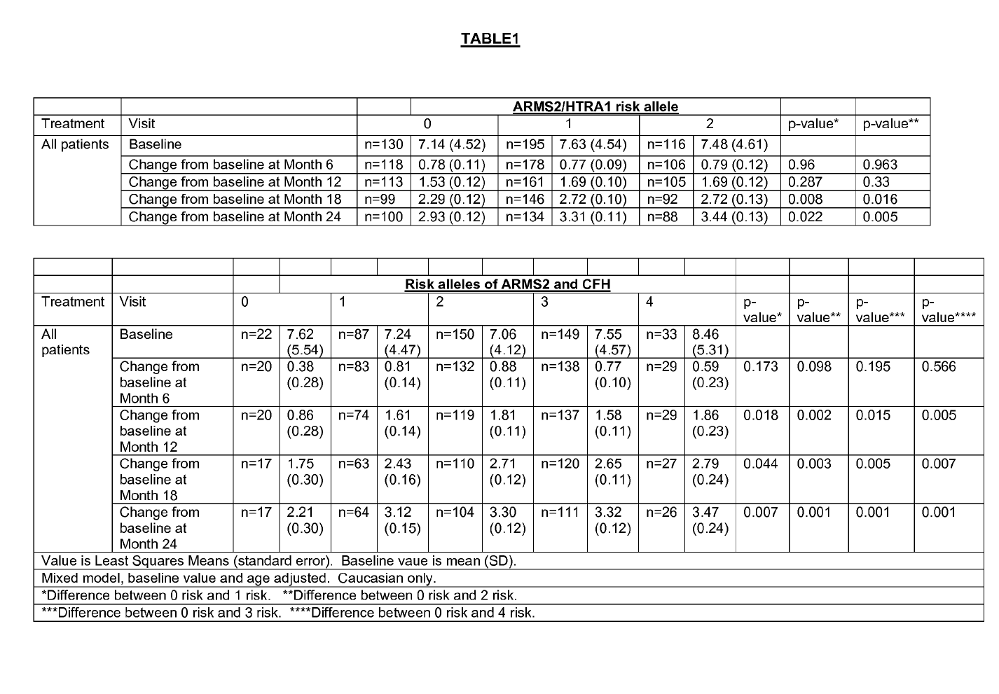

Finally, FIG. 6 shows that patients treated with anti-C5 antibody gained in visual acuity. The patients were able to read 2 more letters after 6 months of treatment with the antibody than those that were sham treated. Impact of GA lesion growth on visual acuity and visual function varies depending on what area of the retina is affected (Sunness et al. (1999) Ophthamol. 106(9):1768-79). Patients with lesions peripheral to the fovea, which is an area of about 1.8 mm responsible for the most detailed, straight-ahead vision of the macula, can have normal visual acuity, but as the lesion grows and fovea is involved, the visual acuity is severely affected. FIG. 6 shows patients with GA involving the fovea were able to improve visual acuity by administration of the anti-C5 antibody.

TABLE 3
SEQUENCES

| SEQ ID | | |
|---|---|---|
| NO. | Information | Sequence |
| (SEQ ID | Age-related maculopathy | TACAATCAAGGTTTTTTTTTTTTTTCA |
| NO. 1) | susceptibility 2 (ARMS2), | AATCCCTGGGTCTCTGCATTTTTTAA |
| | \|rs10490924\| (SEQ ID NO. 1) | AAGCTTCACAGATGATTTCAATGGAT |
| | | ACTAGGGACCTCTGTTGCCTCCTCT |
| | | GGCAGAGCAGGACTGAGGGGTGGA |
| | | CCCTCCCTGAGACCACCCAACAATT |
| | | CAGGGTGGAGTTATCAGGGCGCCCT |
| | | GACTCCTGGGGGCATTTTTGTGTGA |
| | | CGGGAAAAGACAATGCTCCTGGCTG |
| | | AGTGAGATGGCAGCTGGCTTGGCAA |
| | | GGGGACAGCACCTTTGTCACCACAT |
| | | TATGTCCCTGTACCCTACATGCTGCG |
| | | CCTATACCCAGGACCGATGGTAACT |
| | | GAGGCGGAGGGGAAAGGAGGGCCT |
| | | GAGATGGCAAGTCTGTCCTCCTCGG |
| | | TGGTTCCTGTGTCCTTCATTTCCACT |
| | | CTGCGAGAGTCTGTGCTGGACCCTG |
| | | GAGTTGGTGGAGAAGGAGCCAGTGA |
| | | CAAGCAGAGGAGCAAACTGTCTTTAT |
| | | CACACTCCATGATCCCAGCTTCTAAA |
| | | ATCCACACTGAGCTCTGCTTACCAG |
| | | CCTTCTTCTCTCCTGCTGGAACCCAG |
| | | AGGAGGTTCCAGCAGCCTCAGCACC |
| | | ACCTGACACTGGTAAGAAATGCAGA |
| | | TGATCAGGCCTTACCCCAGACCTATT |
| | | GAATCAGAAATTCTGGAGTGGTGCC |
| | | CTGCAGCTTGCATTTTAACCAGCCTT |
| | | CAGGTGCTTCTGATGCATGCTCAGG |
| | | TTTGAGCACCACTGGCCACAGGGAG |
| | | GCCTAGGCAATTCAGCCTTCCTCTG |
| | | GTTGAATAGCTGGAGAATTGGGAAT |
| | | ATCAGTAAATACTTCCAATGCACCTG |
| | | CTACATGCCAGAAAAAGGAAACAAG |
| | | AAGACGCAGTAGGTCTGAGAAAGTG |
| | | ATGGGGTGAGCAGAAACCCAAAGCT |
| | | TATAGAAGGCCATCTGAGTGGCCCC |
| | | TCAAGCCGGTGAATTGGCTTTAGGG |
| | | TTTACTGAAGGAGGTGGAAACCTCA |
| | | GCCTGCTTCTCGTCCGGGTTGTTAG |
| | | AGGAGTCATTTAGAAA |
| (SEQ ID | HtrA serine peptidase 1 | TGGTGAAAAATCAAAAGAGGGGAAA |
| NO. 2) | (HTRA1) \|rs11200638\| | ACCCCTTTCCCATCTGAGACCGCTC |
| | (SEQ ID NO. 2) | CACCCTCGCCAGTTACGAGCTGCCG |
| | | AGCCGCTTCCTAGGCTCTCTGCGAA |
| | | TACGGACACGCATGCCACCCACAAC |
| | | AACTTTTTAAAAGAATCAGACGTGTG |
| | | AAGGATTCTATTCGAATTACTTCTGC |
| | | TCTCTGCTTTTATCACTTCACTGTGG |
| | | GTCTGGGCGCGGGCTTTCTGCCAGC |
| | | TCCGCGGACGCTGCCTTCGTCCGGC |
| | | CGCAGAGGCCCCGCGGTCAGGGTC |
| | | CCGCGTGCGGGGTACCGGGGGCAG |
| | | AACCAGCGCGTGACCGGGGTCCGC |
| | | GGTGCCGCAACGCCCCGGGTCTGC |
| | | GCAGAGGCCCCTGCAGTCCCTGCCC |
| | | GGCCCAGTCCGAGCTTCCCGGGCG |
| | | GGCCCCCAGTCCGGCGATTTGCAGG |
| | | AACTTTCCCCGGCGCTCCCACGCGA |
| | | AGCCGCCGCAGGGCCCCCTTGCAAA |
| | | GTTCCATTAGTTTGAAGGACGCGAAT |
| | | CT |
| (SEQ ID | Complement factor H (CFH) | ATGCCTCAGTAAGTAAACCTCTGAAC |
| NO. 3) | \|rs1061170\| (SEQ ID NO. 3) | TGCTATATATATGTATAAAACTTTCAA |
| | | AGATCGAAGAAAGGAGAGCACATAA |
| | | GTGATTACACCTGTCTTATGTAACAG |
| | | AAATAGGGCCAAGAAAAGAGTTGTT |
| | | CAAGCAAAGTGACCAAAATAGATCTT |
| | | TTCTATTATGAGGGTTTCTTCTTGAA |
| | | AATCACAGGAGAAATAAATATAGGGA |
| | | CTTTATGAGAATATCTATATAATTTAT |
| | | ACATCTATTAATTATAAAAACTAAAGA |
| | | TAAGTAATATTGAATATTGATATTTCT |
| | | TTTTGTGCAAACCTTTGTTAGTAACTT |
| | | TAGTTCGTCTTCAGTTATACATTATTT |
| | | TTGGATGTTTATGCAATCTTATTTAAA |
| | | TATTGTAAAAATAATTGTAATATACTA |
| | | TTTTGAGCAAATTTATGTTTCTCATTT |
| | | ACTTTATTTATTTATCATTGTTATGGT |
| | | CCTTAGGAAAATGTTATTTTCCTTATT |
| | | TGGAAAATGGATATAATCAAAATCAT |
| | | GGAAGAAAGTTTGTACAGGGTAAAT |
| | | CTATAGACGTTGCCTGCCATCCTGG |
| | | CTACGCTCTTCCAAAAGCGCAGACC |
| | | ACAGTTACATGTATGGAGAATGGCT |
| | | GGTCTCCTACTCCCAGATGCATCCG |
| | | TGTCAGTAAGTACACTACTCTGAAAT |
| | | CCTAGCATGTTCATGTCTTTCTAAGT |
| | | AACATAGATGACATTCTAAGACTCAT |
| | | CTATATTAATTGTGGCAAAATGTTTAT |
| | | GTCACCTTGTTTTACCAATGGACCTA |
| | | TTTAGTTTTTGGTTTTTCAACTGTGTA |
| | | TAAATGAATATACAAATTTCTTGATAA |
| | | GTACATAGTAAAATAAATTCTCCTATT |
| | | AATGGGCATTAGTCAAGAATACAGTA |
| | | AAAGAATTTGAACACAATACTTGTTG |
| | | GTTAAATGAAGTCGTATTGAAGCGG |
| | | CATCATTGTCTGGGTAAATATCTGAG |
| | | GTTCGTTATCTCACACCAAGAAGATT |
| | | AAGGACGTGGACACACACAAGGAGT |
| | | GAGTTTA |
| (SEQ ID | Antibody 8109 HCDR1 | SYAIS |
| NO. 4) | | |
| (SEQ ID | Antibody 8109 HCDR2 | GIGPFFGTANYAQKFQG |
| NO. 5) | | |
| (SEQ ID | Antibody 8109 HCDR3 | DTPYFDY |
| NO. 6) | | |
| (SEQ ID | Antibody 8109 LCDR1 | SGDSIPNYYVY |
| NO. 7) | | |
| (SEQ ID | Antibody 8109 LCDR2 | DDSNRPS |
| NO. 8) | | |
| (SEQ ID | Antibody 8109 LCDR3 | QSFDSSLNAEV |
| NO. 9) | | |

1. A method of diagnosing the progression of geographic atrophy (GA) in a subject, the method comprising:
(a) obtaining a sample from a subject;
(b) screening the sample for risk alleles selected from alleles in the group of genes consisting of ARMS2, HTRA1 and CFH;
(c) comparing the results of the screening to the results from a screening of an allele control sample; and
(d) determining that the presence of the risk alleles in the sample indicates an increase in the subject's risk of progression of GA, wherein either:
(i) the presence of at least two ARMS2 risk alleles is determined to be indicative of the progression of GA in the subject; or
(ii) the presence of at least three risk alleles selected from the group of genes consisting of ARMS2 and CFH is determined to be indicative of the progression of GA in the subject.

2. The method of claim 1, wherein the presence of at least two ARMS2 risk alleles is determined to be indicative of the progression of GA in the subject.

3. The method of claim 2, wherein the ARMS2 risk allele comprises rs10490924 (SEQ ID NO. 1).

4. The method of claim 1, wherein the presence of at least three risk alleles selected from the group of genes consisting of ARMS2 and CFH is determined to be indicative of the progression of GA in the subject.

5. The method of claim 4, wherein the ARMS2 risk allele comprises rs10490924 (SEQ ID NO. 1) for ARMS2 and rs1061170 (SEQ ID NO.3) for CFH.

6. The method of claim 1, wherein (d) is:
determining that the presence of the risk alleles in the sample indicates an increase in the subject's risk of progression of GA, wherein either:
(i) the presence of at least two HTRA1 (SEQ ID NO.2) risk alleles is determined to be indicative of the progression of GA in the subject; or
(ii) the presence of at least three risk alleles selected from the group of genes consisting of HTRA1 and CFH is determined to be indicative of the progression of GA in the subject.

7. The method of claim 1, wherein the subject's sample is selected from the group consisting of blood sample, blood-derived product, buccal swab, hair root and leukocyte sample.

8. The method of claim 7, wherein the subject has GA.

9. The method of claim 1, wherein the screening step (b) is a single nucleotide polymorphism (SNP) assay.

10. The method of claim 1, wherein the control sample is from one or more subjects without GA.

11. A method of treating a subject with geographic atrophy (GA), the method comprising;
(a) obtaining a sample from a subject with GA;
(b) screening the sample for the presence of risk alleles selected from alleles in the group of genes consisting of ARMS2 and CFH, wherein the presence of the risk alleles is indicative of the subject's responsiveness to an anti-C5 antibody;
(c) comparing the results of the screening to a control sample;
(d) determining whether the subject's GA will be reduced by the administration of an anti-C5 antibody, wherein:
(i) the presence of at least two ARMS2 risk alleles is indicative of the subject's responsiveness to an anti-C5 antibody; or
(ii) the presence of at least three risk alleles selected from the group of genes consisting of ARMS2 and CFH is indicative of the subject's responsiveness to an anti-C5 antibody; and
(e) administering the anti-C5 antibody to the subject.

12. The method of claim 11, wherein the anti-C5 antibody is antibody 8109 (SEQ ID NOs 4-9).

13. The method of claim 11, wherein the anti-C5 antibody is administered intravitreally to the subject.

14. The method of claim 11, wherein the presence of at least two ARMS2 risk alleles is indicative of subject's responsiveness to anti-C5 antibodies.

15. The method of claim 14, wherein the ARMS2 risk allele comprises rs10490924 (SEQ ID NO. 1).

16. The method of claim 11, wherein the presence of at least three risk alleles of ARMS2 and CFH is indicative of the subject's responsiveness to anti-C5 antibodies.

17. The method of claim 16, wherein the ARMS2 risk allele comprises rs10490924 (SEQ ID NO. 1) for the ARMS2 gene and rs1061170 (SEQ ID NO. 3) for the CFH gene.

18. A composition comprising an anti-C5 antibody for use in treatment of progression of geographic atrophy (GA) in a subject with GA, wherein the subject is selected for anti-C5 antibody treatment by the presence of two ARMS2 risk alleles (SEQ ID NO. 1).

19. A composition comprising an anti-C5 antibody for use in treatment of progression of geographic atrophy in a subject with GA, wherein the subject is selected for anti-C5 antibody treatment by the presence of at least three risk alleles in ARMS2 (SEQ ID NO. 1) and CFH (SEQ ID NO. 3).

20. The composition of claim 19, wherein the anti-C5 antibody is antibody 8109 (SEQ ID NOs. 4-9).
